(19) United States
(12) Patent Application Publication
(10) Pub. No.: US 2013/0164235 A1
(43) Pub. Date: Jun. 27, 2013

(54) COSMETIC COMPOSITION COMPRISING AT LEAST ONE ORGANOPOLYSILOXANE ELASTOMER AND AT LEAST ONE TACKIFYING RESIN

(71) Applicants: Caroline Lebre-Lemonnier, Paris (FR); Christophe Dumousseaux, Antony (FR)

(72) Inventors: Caroline Lebre-Lemonnier, Paris (FR); Christophe Dumousseaux, Antony (FR)

(73) Assignee: L'OREAL, Paris (FR)

(21) Appl. No.: 13/807,987

(22) PCT Filed: Jul. 4, 2011
(86) PCT No.: PCT/EP11/61220

§ 371 Date: Mar. 15, 2013

Related U.S. Application Data
(60) Provisional application No. 61/414,445, filed on Nov. 17, 2010.

(30) Foreign Application Priority Data
Jul. 2, 2010 (FR) 1055369

(51) Int. Cl. A61K 8/81; A61Q 1/10; A61Q 1/02; A61Q 1/06

(52) U.S. Cl. CPC A61K 8/81; A61Q 1/06; A61Q 1/10; A61Q 1/02
USPC 424/63; 424/64; 424/70.7

(57) ABSTRACT

The invention relates to a cosmetic composition for caring for and/or making up keratin materials, comprising, in a physiologically acceptable medium: (i) at least one tackifying resin; (ii) at least one organopolysiloxane elastomer; and (iii) at least one liquid fatty phase.

The invention relates to a cosmetic composition for caring for or making up keratin materials, comprising at least one organopolysiloxane elastomer, one tackifying resin and one liquid fatty phase.

Such a composition exhibits both an easy application and good wear property when it is applied, especially as a thick layer, to keratin materials.

The expression ‘keratin materials’, is understood according to the invention to mean in particular the skin, lips and eyelashes. According to one particular embodiment, it will refer to the skin.

The expression ‘thick layer’, is especially understood according to the invention to mean a deposition with an amount applied of greater than 2 mg/cm², especially ranging from 3 to 200 mg/cm², preferably from 5 to 100 mg/cm².

Care or makeup compositions, when they are applied to the keratin materials, often have the drawback of changing over time, for example in terms of sheen or colour, or even of degrading with a loss of cohesion.

This change may be due to the appearance of sweat or sebum, but also to the mechanical stresses to which the keratin materials are subjected. The sensitivity to mechanical stresses and the poor staying power over time are generally exacerbated when the composition is deposited as a thick layer and on moveable areas: wrinkles of the lips, of the forehead or around the mouth, eyelashes, eyelids.

To improve the wear property of the cosmetic compositions, it is possible to use film-forming and adherent resins or polymers.

The use of a tackifying resin for improving the wear property of foundations is especially known from patent application EP 1 743 626. The compositions described are very fluid, they are deposited on the skin in the form of a thin film with evaporation of a large proportion of the composition. Although these compositions have wear and transfer-resistance properties, they are not suitable for producing thick layer depositions that exhibit good wear property.

There therefore remains a need to have a cosmetic composition that is applied easily, especially as a thick layer and that exhibits good wear property over time for very varied applications such as making up the complexion, lips or eyelashes. Such compositions may especially be suitable for filling imperfections in particular recessed areas of the skin, such as pores, wrinkles or scars.

The term “filling” is intended to mean, not simply depositing a layer which moulds the profile of the imperfections, ie recessed areas in particular of the skins and which fills only the bottom thereof, but, on the contrary, filling at least a large part (typically 50% or more), preferably to the brim, of the recessed area to be masked, in particular wrinkles, pores or scars.

The applicant has now found, surprisingly, that the use, in a cosmetic composition, of an organopolysiloxane elastomer and of a tackifying resin made it possible to obtain compositions that are applied easily, especially as a thick layer and that exhibit good wear property over time.

The present invention therefore relates to a cosmetic composition for caring for and/or making up keratin materials, comprising, in a physiologically acceptable medium:
(i) at least one tackifying resin;
(ii) at least one organopolysiloxane elastomer; and
(iii) at least one liquid fatty phase.

The invention also relates to a cosmetic method for making up and/or non-therapeutically treating keratin materials, in particular the skin, lips or eyelashes, comprising the application to said keratin materials of at least one layer of a composition according to the invention.

In particular, the consumer will be able to apply, to the keratin materials, a deposition with an amount applied of greater than 2 mg/cm², especially ranging from 3 to 200 mg/cm², preferably from 5 to 100 mg/cm², and nevertheless with good wear property over time.

### Organopolysiloxane Elastomer

The organopolysiloxane elastomer makes it possible to thicken the composition and to improve the application properties thereof. It provides a very soft and mattifying feel after application, which is especially advantageous for an application to the skin, in particular for foundation compositions. It may also allow an effective filling of recesses present in the keratin materials.

The expression “organopolysiloxane elastomer” or “silicone elastomer” means a flexible, deformable organopolysiloxane having viscoelastic properties and especially the consistency of a sponge or a flexible sphere. Its modulus of elasticity is such that this material withstands deformation and has limited stretchability and contractability. This material is capable of regaining its original shape after stretching.

It is more particularly a crosslinked organopolysiloxane elastomer.

Thus, the organopolysiloxane elastomer may be obtained by crosslinking addition reaction of diorganopolysiloxane containing at least one hydrogen bonded to silicon and of diorganopolysiloxane containing ethylenically unsaturated groups bonded to silicon, especially in the presence of a platinum catalyst; or by dehydrogenation crosslinking condensation reaction between a diorganopolysiloxane containing hydroxyl end groups and a diorganopolysiloxane containing at least one hydrogen bonded to silicon, especially in the presence of an organotin; or by crosslinking condensation reaction of a diorganopolysiloxane containing hydroxyl end groups and of a hydrolysable organopolysilane; or by thermal crosslinking of organopolysiloxane, especially in the presence of an organoperoxide catalyst; or by crosslinking of organopolysiloxane via high-energy radiation such as gamma rays, ultraviolet rays or an electron beam.

Preferably, the organopolysiloxane elastomer is obtained by crosslinking addition reaction (A) of diorganopolysiloxane containing at least two hydrogens each bonded to a silicon, and (B) of diorganopolysiloxane containing at least two ethylenically unsaturated groups bonded to silicon, especially in the presence (C) of a platinum catalyst, as described, for instance, in patent application EP-A-295 886.

In particular, the organopolysiloxane elastomer may be obtained by reaction of a dimethylpolysiloxane containing dimethylvinylsiloxy end groups and of methylhydrogenpolysiloxane containing trimethylsiloxy end groups, in the presence of a platinum catalyst.

Compound (A) is the base reagent for the formation of organopolysiloxane elastomer, and the crosslinking is performed by addition reaction of compound (A) with compound (B) in the presence of the catalyst (C).

Compound (A) is in particular an organopolysiloxane containing at least two hydrogen atoms bonded to different silicon atoms in each molecule.

Compound (A) may have any molecular structure, especially a linear-chain or branched-chain structure or a cyclic structure.

Compound (A) may have a viscosity at 25° C. ranging from 1 to 50 000 centistokes, especially so as to be miscible with compound (B).

The organic groups bonded to the silicon atoms of compound (A) may be alkyl groups such as methyl, ethyl, propyl, butyl, octyl; substituted alkyl groups such as 2-phenylethyl, 2-phenylpropyl or 3,3,3-trifluoropropyl; aryl groups such as phenyl, tolyl, xylyl; substituted aryl groups such as phenylethyl; and substituted monovalent hydrocarbon-based groups such as an epoxy group, a carboxylate ester group or a mercapto group.

Compound (A) may thus be chosen from methylhydrogenpolysiloxanes containing trimethylsiloxy end groups, dimethylsiloxane-methylhydrosiloxane copolymers containing trimethylsiloxy end groups, and dimethylsiloxane-methylhydrosiloxane cyclic copolymers.

Compound (B) is advantageously a diorganopolysiloxane containing at least two lower alkenyl groups (for example C₂-C₄); the lower alkenyl group may be chosen from vinyl, allyl and propenyl groups. These lower alkenyl groups may be located in any position of the organopolysiloxane molecule, but are preferably located at the ends of the organopolysiloxane molecule. The organopolysiloxane (B) may have a branched-chain, linear-chain, cyclic or network structure, but the linear-chain structure is preferred. Compound (B) may have a viscosity ranging from the liquid state to the gum state. Preferably, compound (B) has a viscosity of at least 100 centistokes at 25° C.

Besides the abovementioned alkenyl groups, the other organic groups bonded to the silicon atoms in compound (B) may be alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

The organopolysiloxanes (B) may be chosen from methylvinylpolysiloxanes, methylvinylsiloxane-dimethylsiloxane copolymers, dimethylpolysiloxanes containing dimethylvinylsiloxy end groups, dimethylsiloxane-methylphenylsiloxane copolymers containing dimethylvinylsiloxy end groups, dimethylsiloxane-diphenylsiloxane-methylvinylsiloxane copolymers containing dimethylvinylsiloxy end groups, dimethylsiloxane-methylvinylsiloxane copolymers containing trimethylsiloxy end groups, dimethylsiloxane-methylphenylsiloxane-methylvinylsiloxane copolymers containing trimethylsiloxy end groups, methyl(3,3,3-trifluoropropyl)polysiloxanes containing dimethylvinylsiloxy end groups, and dimethylsiloxane-methyl(3,3,3-trifluoropropyl)siloxane copolymers containing dimethylvinylsiloxy end groups.

In particular, the organopolysiloxane elastomer may be obtained by reaction of dimethylpolysiloxane containing dimethylvinylsiloxy end groups and of methylhydrogenpolysiloxane containing trimethylsiloxy end groups, in the presence of a platinum catalyst.

Advantageously, the sum of the number of ethylenic groups per molecule in compound (B) and of the number of hydrogen atoms bonded to silicon atoms per molecule in compound (A) is at least 5.

It is advantageous for compound (A) to be added in an amount such that the molecular ratio between the total amount of hydrogen atoms bonded to silicon atoms in compound (A) and the total amount of all the ethylenically unsaturated groups in compound (B) is within the range from 1.5/1 to 20/1.

Compound (C) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

The catalyst (C) is preferably added in an amount of from 0.1 to 1000 parts by weight and better still from 1 to 100 parts by weight, as clean platinum metal, per 1000 parts by weight of the total amount of compounds (A) and (B).

This elastomer may be emulsifying or non-emulsifying. Preferably, it will be non-emulsifying.

The elastomer is advantageously a non-emulsifying elastomer.

The term “non-emulsifying” defines organopolysiloxane elastomers not containing any hydrophilic chains, and in particular not containing any polyoxyalkylene units (especially polyoxyethylene or polyoxypropylene) or any polyglyceryl units. Thus, according to one particular embodiment of the invention, the composition comprises an organopolysiloxane elastomer that is free of polyoxyalkylene units and polyglyceryl units.

The organopolysiloxane elastomer particles are conveyed in the form of a gel formed from an elastomeric organopolysiloxane included in at least one hydrocarbon-based oil and/or one silicone oil. In these gels, the organopolysiloxane particles are often non-spherical particles.

Non-emulsifying elastomers are especially described in patents EP 242 219, EP 285 886 and EP 765 656 and in patent application JP-A-61-194 009.

Non-emulsifying elastomers that may be used more particularly include those sold under the names KSG-6, KSG-15, KSG-16, KSG-18, KSG-41, KSG-42, KSG-43 and KSG-44 by the company Shin-Etsu, DC 9040 and DC 9041 by the company Dow Corning, and SFE 839 by the company General Electric.

Spherical non-emulsifying elastomers that may be used include those sold under the names DC 9040, DC 9041, DC 9509, DC 9505 and DC 9506 by the company Dow Corning.

The elastomer may also be an emulsifying elastomer.

The term “emulsifying organopolysiloxane elastomer” means an organopolysiloxane elastomer comprising at least one hydrophilic chain, such as polyoxyalkylenated organopolysiloxane elastomers and polyglycerolated silicone elastomers.

The emulsifying organopolysiloxane elastomer may be chosen from polyoxyalkylenated organopolysiloxane elastomers.

The polyoxyalkylenated organopolysiloxane elastomer is a crosslinked organopolysiloxane elastomer that may be obtained by crosslinking addition reaction of diorganopolysiloxane containing at least one hydrogen bonded to silicon and of a polyoxyalkylene containing at least two ethylenically unsaturated groups.

Preferably, the polyoxyalkylenated organopolysiloxane elastomer is obtained by crosslinking addition reaction (A1) of diorganopolysiloxane containing at least two hydrogens each bonded to a silicon, and (B1) of polyoxyalkylene containing at least two ethylenically unsaturated groups, especially in the presence (C1) of a platinum catalyst, as described, for instance, in U.S. Pat. No. 5,236,986 and U.S. Pat. No. 5,412,004.

In particular, the organopolysiloxane may be obtained by reaction of polyoxyalkylene (especially polyoxyethylene and/or polyoxypropylene) containing dimethylvinylsiloxy end groups and of methylhydrogenpolysiloxane containing trimethylsiloxy end groups, in the presence of a platinum catalyst.

The organic groups bonded to the silicon atoms of compound (A1) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenylethyl; and substituted monovalent hydrocarbon-based groups such as an epoxy group, a carboxylate ester group or a mercapto group.

Compound (A1) may thus be chosen from methylhydrogenpolysiloxanes containing trimethylsiloxy end groups, dimethylsiloxane-methylhydrosiloxane copolymers containing trimethylsiloxy end groups, dimethylsiloxane-methylhydrosiloxane cyclic copolymers, dimethylsiloxane-methylhydrosiloxane-laurylmethylsiloxane copolymers containing trimethylsiloxy end groups.

Compound (C1) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

Advantageously, the polyoxyalkylenated organopolysiloxane elastomers may be formed from divinyl compounds, in particular polyoxyalkylenes containing at least two vinyl groups, which react with Si—H bonds of a polysiloxane.

Polyoxyalkylenated elastomers are especially described in U.S. Pat. No. 5,236,986, U.S. Pat. No. 5,412,004, U.S. Pat. No. 5,837,793 and U.S. Pat. No. 5,811,487.

Polyoxyalkylenated organopolysiloxane elastomers that may be used include those sold under the names KSG-21, KSG-20, KSG-30, KSG-31, KSG-32, KSG-33, KSG-210, KSG-310, KSG-320, KSG-330 and KSG-340 by the company Shin-Etsu, and DC 9010 and DC 9011 by the company Dow Corning.

The emulsifying organopolysiloxane elastomer may also be chosen from polyglycerolated organopolysiloxane elastomers.

The polyglycerolated organopolysiloxane elastomer according to the invention is an organopolysiloxane elastomer that may be obtained by crosslinking addition reaction of diorganopolysiloxane containing at least one hydrogen bonded to silicon and of polyglycerolated compounds containing ethylenically unsaturated groups, especially in the presence of a platinum catalyst.

Preferably, the organopolysiloxane elastomer is obtained by crosslinking addition reaction (A2) of diorganopolysiloxane containing at least two hydrogens each bonded to a silicon, and (B2) of glycerolated compounds containing at least two ethylenically unsaturated groups, especially in the presence (C2) of a platinum catalyst.

In particular, the organopolysiloxane may be obtained by reaction of a polyglycerolated compound containing dimethylvinylsiloxy end groups and of methylhydrogenpolysiloxane containing trimethylsiloxy end groups, in the presence of a platinum catalyst.

Compound (A2) is the base reagent for the formation of an organopolysiloxane elastomer, and the crosslinking is performed by addition reaction of compound (A2) with compound (B2) in the presence of the catalyst (C2).

Compound (A2) is in particular an organopolysiloxane containing at least two hydrogen atoms bonded to different silicon atoms in each molecule.

Compound (A2) may have any molecular structure, especially a linear-chain or branched-chain structure or a cyclic structure.

Compound (A2) may have a viscosity at 25° C. ranging from 1 to 50 000 centistokes, especially so as to be miscible with compound (B2).

The organic groups bonded to the silicon atoms in compound (A2) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. Preferably, said organic group is chosen from methyl, phenyl and lauryl groups.

Compound (A2) may thus be chosen from methylhydrogenpolysiloxanes containing trimethylsiloxy end groups, dimethylsiloxane-methylhydrosiloxane copolymers containing trimethylsiloxy end groups, dimethylsiloxane-methylhydrosiloxane cyclic copolymers and dimethylsiloxane-methylhydrosiloxane-laurylmethylsiloxane copolymers containing trimethylsiloxy end groups.

Compound (B2) may be a polyglycerolated compound corresponding to formula (B′) below:

CₘH₂ₘ₋₁—O-[Gly]ₙ-CₘH₂ₘ₋₁ (B′)

in which m is an integer ranging from 2 to 6, n is an integer ranging from 2 to 200, preferably from 2 to 100, preferably ranging from 2 to 50, preferably ranging from 2 to 20, preferably ranging from 2 to 10 and preferentially ranging from 2 to 5, and in particular n is equal to 3; Gly denotes:

—CH₂—CH(OH)—CH₂—O— or —CH₂—CH(CH₂OH)—O—

Advantageously, the sum of the number of ethylenic groups per molecule in compound (B2) and of the number of hydrogen atoms bonded to silicon atoms per molecule in compound (A2) is at least 4.

It is advantageous for compound (A2) to be added in an amount such that the molecular ratio between the total amount of hydrogen atoms bonded to silicon atoms in compound (A2) and the total amount of all the ethylenically unsaturated groups in compound (B2) is within the range from 1/1 to 20/1.

Compound (C2) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

The catalyst (C2) is preferably added in an amount of from 0.1 to 1000 parts by weight and better still from 1 to 100 parts by weight, as clean platinum metal, per 1000 parts by weight of the total amount of compounds (A2) and (B2).

The polyglycerolated organopolysiloxane elastomer according to the invention is conveyed in gel form in at least one hydrocarbon-based oil and/or one silicone oil. In these gels, the polyglycerolated elastomer is often in the form of non-spherical particles.

Polyglycerolated organopolysiloxane elastomers that may be used include those sold under the names KSG-710, KSG-810, KSG-820, KSG-830 and KSG-840 by the company Shin-Etsu.

Emulsifying elastomers that may more particularly be used include those sold under the names KSG-31, KSG-32, KSG-33, KSG-210 and KSG-710 by the company Shin-Etsu.

Non-emulsifying elastomers that may be used more particularly include those sold under the names KSG-6, KSG-15, KSG-16, KSG-18, KSG-41, KSG-42, KSG-43 and KSG-44 by the company Shin-Etsu, DC 9040 and DC 9041 by the company Dow Corning, and SFE 839 by the company General Electric.

Preferably, the silicone elastomer is non-emulsifying, in particular in the case of anhydrous compositions.

This organopolysiloxane elastomer is present in the composition generally in a content ranging from 1% to 30% by weight (of active material=dry matter) and preferably from 2% to 10% by weight relative to the total weight of said composition.

### Tackifying Resin

This tackifying resin makes it possible to improve the adhesion of the deposition of the composition to the keratin materials, in particular the skin, and also the cohesion of the film formed. It has little affinity with the organopolysiloxane elastomer, which improves its effectiveness.

Such a resin generally has a number-average molecular weight of less than or equal to 10 000 g/mol, especially ranging from 250 to 10 000 g/mol, especially less than or equal to 5000 g/mol, especially ranging from 250 to 5000 g/mol, in particular less than or equal to 2000 g/mol, especially ranging from 250 to 2000 g/mol and better still less than or equal to 1000 g/mol, more particularly still ranging from 250 to 1000 g/mol.

The number-average molecular weights (Mn) are determined by gel permeation liquid chromatography (THF solvent, calibration curve established with linear polystyrene standards, refractometric detector).

This resin is advantageously a resin as described in the Handbook of Pressure Sensitive Adhesive Technology, edited by Donatas Satas, 3rd ed., 1989, p. 609-619.

It is advantageously chosen from rosin, rosin derivatives, hydrocarbon-based resins and mixtures thereof.
Rosin is a mixture predominantly comprising organic acids known as rosin acids (mainly acids of abietic type and of pimaric type).

There are three types of rosin: rosin (“gum rosin”) obtained by incision on live trees, wood rosin, which is extracted from pine wood or stumps, and tall oil (“tall oil rosin”), which is obtained from a by-product originating from the production of paper.

The rosin derivatives may be derived in particular from the polymerization, hydrogenation and/or esterification (for example with polyhydric alcohols such as ethylene glycol, glycerol or pentaerythritol) of rosin acids. Examples that may be mentioned include the rosin esters sold under the reference Foral 85, Pentalyn H and Staybelite Ester 10 by the company Hercules; Sylvatac 95 and Zonester 85 by the company Arizona Chemical, or Unirez 3013 by the company Union Camp.
The hydrocarbon-based resins are chosen from indene hydrocarbon-based resins, aliphatic pentanediene resins, mixed resins of pentanediene and of indene, diene resins of cyclopentadiene dimers, diene resins of isoprene dimers and mixtures thereof.
   Specifically, the hydrocarbon-based resins are chosen from low molecular weight polymers that may be classified, according to the type of monomer they comprise, as:
indene hydrocarbon-based resins such as resins resulting from the polymerization, in the majority proportion, of indene monomers and, in the minority proportion, of monomers chosen from styrene, methylindene, methylstyrene and mixtures thereof. These resins may optionally be hydrogenated. These resins may have a molecular weight ranging from 290 to 1150 g/mol.

Examples of indene resins that may be mentioned include those sold under the reference Escorez 7105 by the company Exxon Chem., Nevchem 100 and Nevex 100 by the company Neville Chem., Norsolene S105 by the company Sartomer, Picco 6100 by the company Hercules and Resinall by the company Resinall Corp., or the hydrogenated indene/methylstyrene/styrene copolymers sold under the name “Regalite” by the company Eastman Chemical, in particular Regalite R1100, Regalite R1090, Regalite R-7100, Regalite R1010 Hydrocarbon Resin and Regalite R1125 Hydrocarbon Resin.
aliphatic pentanediene resins such as those derived from the polymerization majoritarily of 1,3-pentanediene (trans- or cis-piperylene) monomers and of minority monomers chosen from isoprene, butene, 2-methyl-2-butene, pentene and 1,4-pentanediene, and mixtures thereof. These resins may have a molecular weight ranging from 1000 to 2500 g/mol.
Such 1,3-pentanediene resins are sold, for example, under the references Piccotac 95 by the company Eastman Chemical, Escorez 1304 by the company Exxon Chemicals, Nevtac 100 by the company Neville Chem. or Wingtack 95 by the company Goodyear;
mixed resins of pentanediene and of indene, which are derived from the polymerization of a mixture of pentanediene and indene monomers such as those described above, for instance the resins sold under the reference Escorez 2101 by the company Exxon Chemicals, Nevpene 9500 by the company Neville Chem., Hercotac 1148 by the company Hercules, Norsolene A 100 by the company Sartomer, and Wingtack 86, Wingtack Extra and Wingtack Plus by the company Goodyear;
diene resins of cyclopentadiene dimers such as those derived from the polymerization of a first monomer chosen from indene and styrene, and of a second monomer chosen from cyclopentadiene dimers such as dicyclopentadiene, methyldicyclopentadiene and other pentanediene dimers, and mixtures thereof. These resins generally have a molecular weight ranging from 500 to 800 g/mol, for instance those sold under the reference Betaprene BR 100 by the company Arizona Chemical Co., Neville LX-685-125 and Neville LX-1000 by the company Neville Chem., Piccodiene 2215 by the company Hercules, Petro-Rez 200 by the company Lawter or Resinall 760 by the company Resinall Corp.;
diene resins of isoprene dimers such as terpenic resins derived from the polymerization of at least one monomer chosen from α-pinene, β-pinene and limonene, and mixtures thereof. These resins may have a molecular weight ranging from 300 to 2000 g/mol. Such resins are sold, for example, under the names Piccolyte A115 and S125 by the company Hercules, and Zonarez 7100 or Zonatac 105 Lite by the company Arizona Chem.

Mention may also be made of certain modified resins such as hydrogenated resins, for instance those sold under the name Eastotac C6-C20 Polyolefin by the company Eastman Chemical Co., under the reference Escorez 5300 by the company Exxon Chemicals, or the resins Nevillac Hard or Nevroz sold by the company Neville Chem., the resins Piccofyn A-100, Piccotex 100 or Piccovar AP25 sold by the company Hercules or the resin SP-553 sold by the company Schenectady Chemical Co.

According to one preferred embodiment, the resin is chosen from indene hydrocarbon-based resins, in particular the hydrogenated indene/methylstyrene/styrene copolymers sold under the name “Regalite” by the company Eastman Chemical, such as Regalite R1100, Regalite R1090, Regalite R-7100, Regalite R1010 Hydrocarbon Resin and Regalite R1125 Hydrocarbon Resin.

The tackifying resin is present in the composition in a content ranging generally from 1% to 30% by weight (of active material), preferably from 3% to 20% by weight and preferably from 5% to 15% by weight relative to the total weight of said composition.

It is possible to use, in combination, a block polymer of polystyrene/polyalkylene type, a polyamide silicone block polymer or a dextrin palmitate in order to strengthen the cohesion.

The weight ratio (by weight of active material) between the organopolysiloxane elastomer and the tackifying resin ranges from 0.1 to 10, preferably from 0.2 to 5, preferably from 0.5 to 2.

### Fatty Phase

The composition advantageously contains at least one liquid fatty phase comprising at least one oil, especially chosen from volatile oils, non-volatile oils, and mixtures thereof. The liquid fatty phase will be present in the composition of the invention in a content generally ranging from 1% to 70% by weight, preferably from 10% to 60% by weight and preferably from 20% to 50% by weight relative to the total weight of said composition.

In particular, the cosmetic composition according to the invention is characterized in that the liquid fatty phase comprises at least one hydrocarbon-based oil, in particular a non-volatile hydrocarbon-based oil.

The tackifying resin will preferably be used in a hydrocarbon-based solvent. The composition will therefore contain at least one hydrocarbon-based oil in the liquid fatty phase.

### Oils

The term “oil” means any fatty substance that is in liquid form at room temperature (20-25° C.) and at atmospheric pressure.

The oils may be volatile or non-volatile.

They may be of animal, plant, mineral or synthetic origin.

For the purposes of the present invention, the term “volatile oil” means an oil capable of evaporating on contact with the skin in less than one hour at room temperature and atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg), and preferentially ranging from 1.3 Pa to 1300 Pa (0.01 to 10 mmHg).

For the purposes of the present invention, the term “non-volatile oil” means an oil with a vapour pressure of less than 0.13 Pa.

For the purposes of the present invention, the term “silicone oil” means an oil comprising at least one silicon atom, and especially at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulphur and/or phosphorus atoms, for example in the form of hydroxyl or acid radicals.

### Volatile Oils

The volatile oils may be chosen from hydrocarbon-based oils containing from 8 to 16 carbon atoms, and especially C₈-C₁₆ branched alkanes (also known as isoparaffins), for instance isododecane (also known as 2,2,4,4,6-pentamethylheptane), isodecane and isohexadecane, and for instance the oils sold under the trade names ISOPAR® or PERMETHYL®; volatile linear alkanes, and mixtures thereof.

According to one advantageous embodiment, the volatile linear alkanes that are suitable for the invention have an evaporation rate ranging from 0.01 to 3.5 mg/cm²/minute, at room temperature (25° C.) and atmospheric pressure (760 mmHg), and comprise from 9 to 14 carbon atoms. As examples, mention may be made of n-nonane (C9), n-decane (C10), n-undecane (C11), n-dodecane (C12), n-tridecane (C13) and n-tetradecane (C14), and mixtures thereof. According to one particular embodiment, the volatile linear alkane is chosen from n-nonane, n-undecane, n-dodecane, n-tridecane and n-tetradecane, and mixtures thereof. Preferably, the volatile linear alkane(s) is (are) chosen from n-undecane, n-dodecane, n-tridecane, n-tetradecane, and mixtures thereof. According to one preferred embodiment, the composition according to the invention comprises dodecane. According to another preferred embodiment, the composition according to the invention comprises tetradecane. According to another preferred embodiment, mention may be made of mixtures of n-undecane (C11) and of n-tridecane (C13) obtained in Examples 1 and 2 of patent application WO 2008/155059 of the company Cognis. Mention may also be made of n-dodecane (C12) and n-tetradecane (C14), such as those sold by Sasol under the references, respectively, Parafol 12-97 and Parafol 14-97, and also mixtures thereof.

Volatile oils that may also be used include volatile silicones, for instance volatile linear or cyclic silicone oils, especially those with a viscosity≦8 centistokes (cSt) (8×10⁻⁶ m²/s), and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the invention, mention may be made especially of dimethicones of viscosity 5 and 6 cSt, octamethylcyclotetrasiloxane, decamethylcyclopentasiloxane, dodecamethylcyclohexa-siloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoromethyl-cyclopentane, and mixtures thereof, may also be used.

According to one particular embodiment, the composition is a foundation and comprises at least one volatile silicone oil.

According to one particular embodiment, the composition is a mascara and comprises at least one volatile hydrocarbon-based oil, in particular chosen from isododecane, isohexadecane, and a mixture thereof.

### Non-Volatile Oils

The non-volatile oils may be chosen especially from non-volatile hydrocarbon-based, fluoro and/or silicone oils.

Advantageously, the composition of the invention comprises at least one non-volatile hydrocarbon-based oil.

Non-volatile hydrocarbon-based oils that may especially be mentioned include:
hydrocarbon-based oils of animal origin,
hydrocarbon-based oils of plant origin, such as phytostearyl esters, such as phytostearyl oleate, phytostearyl isostearate and lauroyl/octyldodecyl/phyto-stearyl glutamate (Ajinomoto, Eldew PS203), triglycerides formed from fatty acid esters of glycerol, in particular in which the fatty acids may have chain lengths ranging from C₄ to C₃₆ and especially from C₁₈ to C₃₆, these oils possibly being linear or branched, and saturated or unsaturated; these oils may especially be heptanoic or octanoic triglycerides, shea oil, alfalfa oil, poppy oil, millet oil, barley oil, rye oil, candlenut oil, passionflower oil, shea butter, aloe vera oil, sweet almond oil, peach stone oil, groundnut oil, argan oil, avocado oil, baobab oil, borage oil, broccoli oil, calendula oil, camellina oil, canola oil, carrot oil, safflower oil, flax oil, rapeseed oil, cotton oil, coconut oil, marrow seed oil, wheatgerm oil, jojoba oil, lily oil, macadamia oil, corn oil, meadowfoam oil, St John's Wort oil, monoi oil, hazelnut oil, apricot kernel oil, walnut oil, olive oil, evening primrose oil, palm oil, blackcurrant pip oil, kiwi seed oil, grapeseed oil, pistachio oil, winter squash oil, pumpkin oil, quinoa oil, musk rose oil, sesame oil, soybean oil, sunflower oil, castor oil and watermelon oil, and mixtures thereof, or alternatively caprylic/capric acid triglycerides, such as those sold by the company STÉARINERIES DUBOIS or those sold under the names MIGLYOL 810®, 812® and 818® by the company Dynamit Nobel;
   synthetic ethers containing from 10 to 40 carbon atoms, such as dicapryl ether;
synthetic esters, for instance the oils of formula R₁COOR₂, in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10. The esters may be chosen especially from esters of alcohol and of fatty acid, for instance cetostearyl octanoate, esters of isopropyl alcohol, such as isopropyl myristate, isopropyl palmitate, ethyl palmitate, 2-ethylhexyl palmitate, isopropyl stearate, octyl stearate, hydroxylated esters, for instance isostearyl lactate, octyl hydroxystearate, alcohol or polyalcohol ricinoleates, hexyl laurate, neopentanoic acid esters, for instance isodecyl neopentanoate, isotridecyl neopentanoate, and isononanoic acid esters, for instance isononyl isononanoate and isotridecyl isononanoate.
polyol esters and pentaerythritol esters, for instance dipentaerythrityl tetrahydroxystearate/tetraisostearate;
fatty alcohols that are liquid at room temperature, with a branched and/or unsaturated carbon-based chain containing from 12 to 26 carbon atoms, for instance 2-octyldodecanol, isostearyl alcohol and oleyl alcohol;

C₁₂-C₂₂ higher fatty acids, such as oleic acid, linoleic acid or linolenic acid, and mixtures thereof;
dialkyl carbonates, the two alkyl chains possibly being identical or different, such as dicaprylyl carbonate sold under the name CETIOL CC®, by Cognis; and
oils of high molar mass, in particular with a molar mass ranging from about 400 to about 2000 g/mol and in particular from about 650 to about 1600 g/mol. As oils of high molar mass that may be used in the present invention, mention may be made especially of linear fatty acid esters with a total carbon number ranging from 35 to 70, for instance pentaerythrityl tetrapelargonate, hydroxylated esters, such as polyglyceryl-2 triisostearate, aromatic esters, such as tridecyl trimellitate, esters of branched C₂₄-C₂₈ fatty alcohols or fatty acids, such as those described in U.S. Pat. No. 6,491,927, and pentaerythritol esters, and especially triisoarachidyl citrate, glyceryl triisostearate, glyceryl tris(2-decyl)tetradecanoate, polyglyceryl-2 tetraisostearate or pentaerythrityl tetrakis(2-decyl)tetradecanoate; phenyl silicones, such as Belsil PDM 1000 from the company Wacker (MM=9000 g/mol), non-volatile polydimethylsiloxanes (PDMS), PDMSs comprising alkyl or alkoxy groups that are pendent and/or at the end of the silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, dimethicones or phenyl trimethicones with a viscosity of less than or equal to 100 cSt, and mixtures thereof; and also mixtures of these various oils.

According to one particular embodiment, the composition according to the invention comprises at least isononyl isononanoate.

### Fatty Phase Structuring or Gelling Agent

To strengthen the rigid nature of the composition and prevent exudation of the liquid fatty phase, a lipophilic structuring or gelling agent may advantageously be added to the composition according to the invention.

The fatty phase structuring or gelling agent will generally be present in the composition in a content ranging from 1% to 30% by weight, preferably from 3% to 20% by weight and preferably from 5% to 15% by weight relative to the total weight of said composition.

### Mineral Lipophilic Gelling Agents

A lipophilic gelling agent may be mineral or organic (especially polymeric).

Examples of mineral lipophilic gelling agents that may be mentioned include hydrophobic modified clays such as modified magnesium silicate (Bentone Gel VS38 from Rheox), or hectorite modified with distearyldimethylammonium chloride (CTFA name: Disteardimonium hectorite) sold under the name Bentone 38 CE by the company Rheox. Mineral lipophilic gelling agents that may be mentioned include optionally modified clays, for instance hectorites modified with a C₁₀ to C₂₂ fatty acid ammonium chloride, for instance hectorite modified with distearyldimethylammonium chloride, for instance the product sold under the name Bentone 38V® by the company Elementis.

### Additional Polymeric Organic Gelling Agents

The polymeric organic lipophilic gelling agents are, for example, ethyl cellulose for instance the product sold under the name Ethocel® by the company Dow Chemical; the polycondensates of polyamide type resulting from the condensation between a dicarboxylic acid comprising at least 32 carbon atoms and an alkylenediamine and in particular ethylenediamine, in which the polymer comprises at least one terminal carboxylic acid group esterified or amidified with at least one monoalcohol or one monoamine comprising from 12 to 30 linear and saturated carbon atoms, and in particular, ethylenediamine/stearyl dilinoleate copolymers such as that sold under the name Uniclear 100 VG® by the company Arizona Chemical; galactomannans comprising from one to six and in particular from two to four hydroxyl groups per saccharide, substituted with a saturated or unsaturated alkyl chain, for instance guar gum alkylated with C₁ to C₆, and in particular C₁ to C₃, alkyl chains, and mixtures thereof.

Mention may also be made of polyamide silicone block polymers also known as silicone polyamides or PSPAs. Silicone polyamides are preferably solid at room temperature (25° C.) and atmospheric pressure (760 mmHg). The silicone polyamides of the composition of the invention may be polymers of polyorganosiloxane type for instance those described in documents U.S. Pat. No. 5,874,069, U.S. Pat. No. 5,919,441, U.S. Pat. No. 6,051,216 and U.S. Pat. No. 5,981,680. According to the invention, the silicone polymers may belong to the following two families:
(1) polyorganosiloxanes comprising at least two amide groups, these two groups being located in the polymer chain, and/or
(2) polyorganosiloxanes comprising at least two amide groups, these two groups being located on grafts or branches.

As lipophilic gelling agents that are suitable for the invention, mention may also be made of copolymers of the polystyrene/polyalkylene type, and more particularly block copolymers of “diblock”, “triblock” or “radial” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Kraton Polymers, or of the polystyrene/copoly(ethylene-butylene) type, and mixtures of triblock and radial (star) copolymers in isododecane, such as those sold by the company Penreco under the name Versagel®, for instance the mixture of butylene/ethylene/styrene triblock copolymer and of ethylene/propylene/styrene star copolymer in isododecane (Versagel M 5960).

Among the lipophilic gelling agents that may be used in a cosmetic composition of the invention, mention may also be made of fatty acid esters of dextrin, such as dextrin palmitates, especially the products sold under the names Rheopearl TL® or Rheopearl KL® by the company Chiba Flour, hydrogenated plant oils such as hydrogenated castor oil, fatty alcohols, in particular C₈ to C₂₆, and more particularly C₁₂ to C₂₂ fatty alcohols, for instance myristyl alcohol, cetyl alcohol, stearyl alcohol or behenyl alcohol.

According to one particular embodiment, the composition according to the invention does not contain polyamide silicone block polymers.

Mention may also be made of organogelling agents, especially chosen from those described in patent application WO-A-03/105788.

Examples that may be mentioned include

bis-urea derivatives of general formula (II):

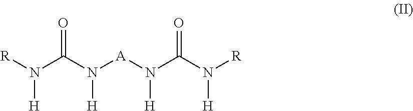

in which:
   A is a group of formula:

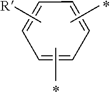

with R′ being a linear or branched C₁ to C₄ alkyl radical, and the *s symbolizing the points of attachment of the group A to each of the two nitrogen atoms of the rest of the compound of general formula (I), and
R is a saturated or unsaturated, non-cyclic, monobranched C₆ to C₁₅ alkyl radical, the hydrocarbon-based chain of which is optionally interrupted by 1 to 3 heteroatoms chosen from O, S and N, or
a salt or isomer thereof in particular described in patent application FR-A-2892303;

silicone bis-urea derivatives of general formula (III) or a salt and/or isomer thereof:

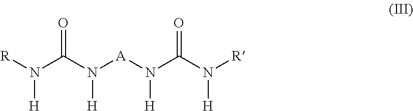

in which:
   A is a group of formula (IV):

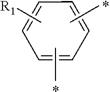

with R1 being a linear or branched C₁ to C₄ alkyl radical, and the *s symbolizing the points of attachment of the group A to each of the two nitrogen atoms of the rest of the compound of general formula (I), and
R and R′, which may be identical or different, are chosen from:
i) radicals of formula (V):

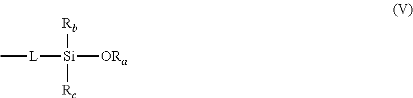

in which:
   L is a single bond or a divalent carbon-based radical, especially a linear, branched and/or cyclic, saturated or unsaturated hydrocarbon-based radical (alkylene), comprising 1 to 18 carbon atoms, and possibly comprising 1 to 4 heteroatoms chosen from N, O and S;
   Ra is:
a) a carbon-based radical, especially a linear, branched and/or cyclic, saturated or unsaturated hydrocarbon-based radical (alkyl), comprising 1 to 18 carbon atoms, and possibly comprising 1 to 8 heteroatoms chosen from N, O, Si and S; or
b) a silicone radical of formula:

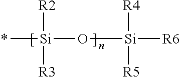

with n being between 0 and 100, especially between 1 and 80, or even 2 to 20;
and R2 to R6 being, independently of each other, carbon-based radicals, especially linear or branched hydrocarbon-based radicals (alkyls) comprising 1 to 12 and especially 1 to 6 carbon atoms, and possibly comprising 1 to 4 heteroatoms, especially O;
   Rb and Rc are, independently of each other, chosen from:
a) carbon-based radicals, especially linear, branched and/or cyclic, saturated or unsaturated hydrocarbon-based radicals (alkyls), comprising 1 to 18 carbon atoms, and possibly comprising 1 to 4 heteroatoms chosen from N, O, Si and S; or
b) radicals of formula:

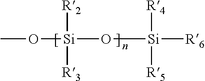

with n being between 0 and 100, especially between 1 and 80, or even 2 to 20;
and R′2 to R′6 being, independently of each other, carbon-based radicals, especially linear or branched hydrocarbon-based radicals (alkyls) comprising 1 to 12 and especially 1 to 6 carbon atoms, and possibly comprising 1 to 4 heteroatoms, especially O;
and
   ii) linear, branched and/or cyclic, saturated or unsaturated C₁ to C₃₀ alkyl radicals, optionally comprising 1 to 3 heteroatoms chosen from O, S, F and N; it being understood that at least one of the R and/or R′ radicals is of formula (III) such as those described in patent application FR-A-2900819.

The bis-urea derivatives described in patent application FR-A-2894476.

According to one particularly preferred embodiment, a composition according to the invention may comprise at least one polymeric organic lipophilic gelling agent, in particular chosen from copolymers of the polystyrene/polyalkylene type, and more particularly copolymers of the polystyrene/copoly(ethylene-propylene) type, especially those sold under the name Kraton® by the company Kraton Polymers.

### Lipophilic Structuring Agents

In particular, said lipophilic structuring agent may be chosen from at least one wax, at least one gum and/or at least one pasty fatty substance, of plant, animal, mineral or synthetic origin, or even silicone origin, and mixtures thereof.

### Waxes

Among the waxes that are solid at room temperature that are capable of being present in the composition according to the invention, mention may be made of hydrocarbon-based waxes such as beeswax; carnauba wax, candelilla wax, ouricury wax, Japan wax, cork fibre wax or sugarcane wax; paraffin wax, lignite wax; microcrystalline waxes; lanolin wax, Montan wax; ozokerites; polyethylene waxes; waxes obtained by Fischer-Tropsch synthesis; hydrogenated oils, fatty esters and glycerides that are solid at 25° C. It is also possible to use silicone waxes, for instance alkyl or alkoxy polymethylsiloxanes and/or polymethylsiloxane esters. The waxes may be in the form of stable dispersions of colloidal wax particles such as may be prepared according to known methods, such as those from “Microemulsions Theory and Practice”, L. M. Prince Ed., Academic Press (1977), pages 21-32. As wax that is liquid at room temperature, mention may be made of jojoba oil.

Mention may also be made of hydrogenated plant oils, such as hydrogenated castor oil.

As lipophilic structuring agent that is also suitable for the invention, mention may be made of fatty alcohols, in particular C₈ to C₂₆, and more particularly C₁₂ to C₂₂, fatty alcohols.

According to one embodiment, a fatty alcohol that is suitable for the invention may be chosen from myristyl alcohol, cetyl alcohol, stearyl alcohol or behenyl alcohol.

As lipophilic structuring agent that is also suitable for the invention, mention may be made of fatty acid esters of glycerols, such as glyceryl tristearate.

According to one particular embodiment, the composition may also contain at least one wax having an initial melting point greater than or equal to 50° C., and better still at least one wax for which the initial melting point is greater than or equal to 65° C.

The expression “initial melting point” is understood in the present application to mean the temperature at which a wax begins to melt. This temperature can be determined by DTA (differential thermal analysis) which makes it possible to obtain the thermogram (or melting curve) of the wax in question. The initial melting point corresponds to the temperature at which a significant change of gradient can be observed in the thermogram. The melting point itself represents the minimum point of said thermogram.

More specifically, such a wax may be chosen from carnauba wax, certain polyethylene waxes and certain microcrystalline waxes such as those sold under the name Microwax®, in particular that sold under the name Microwax HW®, by the company Paramelt.

It may make it possible to thicken and stabilize the composition according to the invention.

The amount of wax will generally range from 0.1% to 25% by weight, preferably from 1% to 15% by weight and preferably from 2% to 12% by weight relative to the total weight of said composition.

### Pasty Fatty Compounds

Pasty fatty compounds can be defined using at least one of the following physicochemical properties:
a viscosity of 0.1 to 40 Pa·s (1 to 400 poise), preferably 0.5 to 25 Pa·s, measured at 40° C. with a CONTRAVES TV rotary viscometer equipped with an MS-r3 or MS-r4 spindle at a frequency of 60 Hz;
a melting point of 25-70° C., preferably 25-55° C.

The compositions of the invention may also comprise at least one alkyldimethicone, alkoxydimethicone or phenyldimethicone such as, for example, the product sold under the name “Abil wax 2440” by the company Goldschmidt.

Thus, the composition according to the invention advantageously also comprises at least one fatty phase structuring or gelling agent, especially chosen from waxes, polymeric thickeners or gelling agents, organogelling agents and mixtures thereof.

In particular, it may especially be chosen from:
1) waxes,
2) polymeric thickeners or gelling agents, for example the silicone elastomers, polyamides, silicone polyamides, block copolymers of Kraton type,
3) organogelling agents.

### Pulverulent Phase

The composition will preferably contain a pulverulent phase, especially comprising fillers and/or colorants and optionally fibres.

### Fillers

The term “fillers” should be understood as meaning colourless or white, mineral or organic particles, which are insoluble in the medium of the composition irrespective of the temperature at which the composition is manufactured, and which do not colour the composition.

The fillers may be of any shape, platelet-shaped, spherical, fibrous, hemispherical or oblong, irrespective of the crystallographic form (for example lamellar, cubic, hexagonal, orthorhombic, etc.).

By way of illustration of these fillers, mention may be made of talc, mica, silica, kaolin, poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie), or of acrylic acid copolymers, silicone resin microbeads (Tospearls® from Toshiba, for example), elastomeric polyorganosiloxane particles, precipitated calcium carbonate, magnesium carbonate, magnesium hydrogen carbonate, hydroxyapatite, barium sulphate, aluminium oxides, polyurethane powders, composite fillers, hollow silica microspheres, and glass or ceramic microcapsules.

Very particularly suitable for the invention, are the fillers chosen from:
porous silica microparticles, for instance the Silica Beads SB150® and SB700® from Miyoshi, with a mean size of 5 microns; H series Sunsphere® products from Asahi Glass, for instance the products Sunsphere H33®, H51®, and H53® having respective sizes of 3, 5 and 5 microns;
polytetrafluoroethylene (PTFE) powders, for instance the PTFE Ceridust 9205F® from Clariant, with a mean size of 8 microns;
silicone resin powders, for instance the silicone resin Tospearl 145A® from GE Silicone, with a mean size of 4.5 microns;
hollow hemispherical silicone particles, for instance NLK 500, NLK 506 and NLK 510 from Takemoto Oil and Fat;
acrylic copolymer powders, especially polymethyl methacrylate (PMMA) powders, for instance the Jurymer MBI® PMMA particles from Nihon Junyoki, with a mean size of 8 microns, hollow PMMA spheres, for instance sold under the name Covabead LH85® by the company Wacker, and vinylidene chloride/acrylonitrile/methylene methacrylate expanded microspheres sold under the name Expancel®;
polyethylene powders, especially comprising at least one ethylene/acrylic acid copolymer, and in particular consisting of ethylene/acrylic acid copolymers, for instance the polyethylene particles AC540® or else the Flobead EA 209® particles from Sumitomo, with a mean size of 10 microns;
elastomeric crosslinked organopolysiloxane powders coated with silicone resin, especially with silsesquioxane resin, as described, for example, in U.S. Pat. No. 5,538,793. Such elastomer powders are especially sold under the names KSP-100®, KSP-101®, KSP-102®, KSP-103®, KSP-104® and KSP-105® by the company Shin Etsu;
talc/titanium dioxide/alumina/silica composite powders for instance those sold under the name Coverleaf AR-80® by the company Catalyst & Chemicals;
   polyamide powders (Nylon®), for instance Nylon-12 particles of the Orgasol® type from Arkema, with a mean size of 10 microns;
   expanded powders such as hollow microspheres and especially the microspheres formed from a vinylidene chloride/acrylonitrile/methacrylate terpolymer, for instance those sold under the name Expancel® by the company Kemanord Plast under the references 551 DE 12® (particle size of around 12 μm and density of 40 kg/m³), 551 DE 20® (particle size of around 30 μm density of 65 kg/m³) and 551 DE 50® (particle size of around 40 μm);
   microspheres sold under the name MICROPEARL F 80 ED® by the company Matsumoto;
   powders of natural organic materials such as starch powders, especially crosslinked or non-crosslinked corn, wheat or rice starch powders, such as the starch powders crosslinked with octenyl succinate anhydride, and especially those sold under the name DRY-FLO® by the company National Starch;
and mixtures thereof.

According to one preferred embodiment, a composition according to the invention comprises at least one filler chosen from silica, porous silica microparticles, silicone resin powders, polyethylene powders, mixtures of crosslinked polydimethylsiloxane and of polydimethylsiloxane, expanded powders and mixtures thereof.

### Fibres

As specified above, a composition according to the invention may comprise fibres, especially at least 5% by weight of fibres, relative to its total weight.

The term “fibre” should be understood as meaning an object of length L and of diameter D such that L is greater than D and preferably very much greater than D, D being the diameter of the circle in which the cross section of the fibre is inscribed. In particular, the ratio L/D (or aspect ratio) is chosen in the range from 3.5 to 2500, preferably from 5 to 500 and better still from 5 to 150.

The fibres that may be used in the composition of the invention may be mineral or organic fibres, of synthetic or natural origin, and they may be flexible or rigid. They may be short or long, individual or organized, for example braided. They may have any shape and may especially have a circular or polygonal (square, hexagonal or octagonal) cross section depending on the specific application envisaged. In particular, their ends are blunted and/or polished to prevent injury.

In particular, the fibres according to the invention preferably have a circular cross section.

In particular, the fibres have a length ranging from 1 μm to 10 mm, preferably from 0.1 mm to 5 mm and better still from 0.1 mm to 3 mm. Their cross section may be included in a circle with a diameter ranging from 2 nm to 500 μm, preferably ranging from 100 nm to 100 μm. The weight of the fibres is often given in denier or decitex.

The fibres may be those used in the manufacture of textiles and especially silk fibres, cotton fibres, wool fibres, flax fibres, cellulose fibres extracted, for example, from wood, from vegetables or from algae, polyamide (Nylon®, especially under the names Nylon 6=polyamide 6; Nylon 6,6 or Nylon 66=polyamide 6,6; Nylon 12=polyamide 12) fibres, rayon fibres, viscose fibres, acetate fibres, in particular rayon acetate fibres, cellulose acetate fibres or silk acetate fibres, poly(p-phenyleneterephthalamide) fibres, acrylic polymer fibres, especially polymethyl methacrylate fibres or poly-2-hydroxyethylmethacrylate fibres, polyolefin fibres and especially polyethylene or polypropylene fibres, glass fibres, silica fibres, carbon fibres, especially in graphite form, polytetrafluoroethylene (such as Teflon®) fibres, insoluble collagen fibres, polyester fibres, polyvinyl chloride fibres or polyvinylidene chloride fibres, polyvinyl alcohol fibres, polyacrylonitrile fibres, chitosan fibres, polyurethane fibres, polyethylene phthalate fibres, and fibres formed from a blend of polymers such as those mentioned above, for instance trilobed polyamide/polyester fibres, and mixtures of these fibres.

Moreover, the fibres may or may not be surface-treated, and may be coated or uncoated. As coated fibres that can be used in the invention, mention may be made of polyamide fibres coated with copper sulphide to give an antistatic effect (for example, R-STAT fibres from the company Rhodia) or fibres coated with another polymer enabling a particular organization of the fibres (specific surface treatment) or a surface treatment inducing colour/hologram effects (Lurex fibre from the company Sildorex, for example).

The fibres that can be used in the composition according to the invention are preferably chosen from polyamide fibres, cellulose fibres and polyethylene fibres, and mixtures thereof. The length thereof may range from 0.1 to 5 mm, preferably from 0.25 to 1.6 mm, and the mean diameter thereof may range from 5 to 50 μm.

Fibres chosen from Nylon 6 (or polyamide 6), Nylon 6,6 or Nylon 66 (or polyamide 6,6) and Nylon 12 (or polyamide 12) fibres, and mixtures thereof, are very particularly suitable.

In particular, use may be made of the polyamide fibres sold by Etablissements P. Bonte under the name polyamide 0.9 dtex 0.3 mm (INCI name: Nylon 6,6), having a mean diameter of 6 μm, a weight of approximately 0.9 dtex and a length ranging from 0.3 mm to 3 mm, or alternatively the polyamide fibres sold under the name Fiberlon 931-D1-S by the company LCW, having a yarn count of approximately 0.9 dtex and a length of approximately 0.3 mm. Use may also be made of Nylon-66 fibres, having a yarn count of approximately 2 dtex, and a length of approximately 0.3 mm, sold under the name “Polyamide brillante trilobée” by the company Utexbel (INCI name: Nylon-66).

Use may also be made of cellulose (or rayon) fibres having an average diameter of 50 μm and a length ranging from 0.5 mm to 6 mm, such as those sold under the name Natural rayon flock fibre RC1BE-N003-M04 by the company Claremont Flock. Use may also be made of polyethylene fibres such as those sold under the name Shurt Stuff 13 099 F by the company Mini Fibers.

### Colorants

A cosmetic composition in accordance with the invention may advantageously incorporate at least one colorant chosen from organic or mineral colorants, especially such as the pigments or nacres conventionally used in cosmetic compositions, liposoluble or water-soluble colorants and mixtures thereof.

The term “pigments” should be understood as meaning white or coloured, mineral or organic particles, which are insoluble in an aqueous solution and which are intended to colour and/or opacify the resulting film.

The pigments may be present in a proportion of from 0.1% to 40% by weight, especially from 1% to 30% by weight and in particular from 5% to 15% by weight relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The colorant may also comprise a pigment having a structure that may be, for example, of silica microsphere type containing iron oxide. An example of a pigment having this structure is the product sold by the company Miyoshi under the reference PC Ball PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542 669, EP-A-787 730, EP-A-787 731 and WO-A-96/08537.

The term “nacres” should be understood as meaning iridescent or non-iridescent coloured particles of any form, especially produced by certain molluscs in their shell, or else synthesized, and which have a colour effect by optical interference.

The nacres may be chosen from nacreous pigments such as titanium mica coated with an iron oxide, titanium mica coated with bismuth oxychloride, titanium mica coated with chromium oxide, titanium mica coated with an organic colorant and also nacreous pigments based on bismuth oxychloride. They may also be mica particles at the surface of which are superposed at least two successive layers of metal oxides and/or of organic colorants.

Examples of nacres that may also be mentioned include natural mica coated with titanium oxide, with iron oxide, with natural pigment or with bismuth oxychloride.

Among the nacres available on the market, mention may be made of the nacres Timica, Flamenco and Duochrome (based on mica) sold by the company Engelhard, the Timiron nacres sold by the company Merck, the Prestige mica-based nacres, sold by the company Eckart, and the Sunshine synthetic mica-based nacres, sold by the company Sun Chemical.

The nacres may more particularly have a yellow, pink, red, bronze, orange, brown, gold and/or coppery color or tint.

The cosmetic composition according to the invention may also comprise water-soluble or liposoluble colorants. The liposoluble colorants are, for example, Sudan Red, DC Red 17, DC Green 6, β-carotene, soybean oil, Sudan Brown, DC Yellow 11, DC Violet 2, DC Orange 5 and quinoline yellow. The water-soluble colorants are, for example, beetroot juice and caramel.

This pulverulent phase will be present in a content generally ranging from 1% to 70% by weight, preferably from 10% to 60% by weight and preferably from 20% to 50% by weight relative to the total weight of said composition.

This pulverulent phase preferably contains fibres.

The weight ratio between the liquid fatty phase and pulverulent phase will advantageously range from 0.1 to 10, preferably from 0.2 to 5 and preferably from 0.5 to 3.

### Galenic Form

The composition according to the invention may be in various forms, especially in the form of an anhydrous composition, a dispersion, an emulsion, such as in particular a water/oil or oil/water or multiple emulsion.

A composition of the invention is preferably an emulsion, in particular a direct or inverse emulsion, or an anhydrous composition.

The composition may thus contain an aqueous phase.

A composition of the invention may comprise an aqueous phase in a content ranging from 1% to 80% by weight, especially from 10% to 80% and more particularly from 20% to 60% by weight, especially from 30% to 50% by weight relative to the total weight of the composition.

The aqueous phase generally comprises water such as: demineralized water, a water from a natural source, such as La Roche-Posay water, a floral water such as cornflower water and/or a spring water.

According to one embodiment, a composition of the invention may also comprise at least one water-miscible, organic solvent.

The water-miscible, organic solvent(s) suitable for the invention may be chosen from C₁₋₈, and especially C₁₋₅, monoalcohols, especially ethanol, isopropanol, tert-butanol, n-butanol, polyols as described previously, and mixtures thereof.

A composition of the invention may also comprise at least one salt, for example sodium chloride, magnesium chloride or magnesium sulphate.

A composition of the invention may comprise from 0.05% to 1.5%, in particular from 0.1% to 1.0% and more particularly from 0.15% to 0.8% by weight of salts relative to the total weight of the composition.

According to another preferred embodiment, the composition of the invention advantageously contains less than 2% by weight of water and preferably less than 1% by weight of water relative to the total weight of the composition, or is anhydrous. The term “anhydrous” especially means that water is preferably not deliberately added to the composition, but may be present in trace amount in the various compounds used in the composition.

Preferably, the composition is anhydrous.

A cosmetic composition according to the invention may also additionally comprise any additive usually used in the field in question, for example chosen from surfactants, thickeners, humectants, hydrophilic or lipophilic active agents, film-forming agents, and where appropriate film-forming auxiliaries, gums, semi-crystalline polymers, antioxidants, vitamins, essential oils, preservatives, fragrances, neutralizers, antiseptic agents, UV-protecting agents, and mixtures thereof.

The composition according to the invention may be produced by known processes, generally used in the cosmetics or dermatological field.

The composition according to the invention may be in the form of a cream or a paste, or be in pulverulent form.

### Preparation Process

A composition according to the invention may be prepared advantageously by using, for at least one step of the process, a mixing device such as a roll mill comprising two rolls revolving in opposite directions, between which the paste passes, or a screw-mixer extruder. A screw-mixer extruder is preferably used.

The rest of the liposoluble ingredients may then be mixed together at a temperature of about 100° C. The ground material or the predispersed active agents may then be added to the oily phase.

The composition is obtained preferably by a high-temperature mixing process or by an extrusion process.

In order to effectively incorporate the waxes and the fillers, it is advantageous to carry out the preparation of the composition or at least one step of this preparation, in particular the preparation of the oily phase or one step of the preparation of the oily phase, in a screw-mixer extruder, subjected to a temperature gradient ranging from 100° C. to 20° C. In particular, it is advantageous to carry out the mixing of the fillers, waxes and at least one oil, in a screw-mixer extruder, subjected to a temperature gradient ranging from 100° C. to 20° C. Such a preparation is especially described in patent applications EP 1 005 856, EP 1 005 857 and EP 1 013 267.

The invention is illustrated in greater detail in the examples that follow, which are given as non-limiting illustrations. The percentages are weight percentages.

### EXAMPLE 1

### Example of Coloured Smoothing Base

| | | Inven- | Compara- |
|---|---|---|---|
| | | tion | tive |
| Phase | INCI name | example | example |
| A | MICROCRYSTALLINE WAX | 6.3 | 6.3 |
| | ETHYLENE/ACRYLIC ACID | 3.1 | 3.1 |
| | COPOLYMER (1) | | |
| | HYDROGENATED STYRENE/METHYL | 8.0 | 0 |
| | STYRENE/INDENE COPOLYMER (2) | | |
| | HYDROGENATED STYRENE/ | 1.0 | 1.0 |
| | BUTADIENE COPOLYMER (3) | | |
| | HYDROGENATED POLYISOBUTENE | 18.9 | 21.9 |
| | DIMETHICONE | 11.7 | 11.7 |
| | ISONONYL ISONONANOATE | 3.8 | 3.8 |
| | DIMETHICONE/VINYL DIMETHICONE | 9.7 | 9.7 |
| | CROSSPOLYMER (and) | | |
| | DIMETHICONE (4) | | |
| B | SILICA (5) | 2.6 | 2.6 |
| | ACRYLATES COPOLYMER (6) | 0.2 | 0.2 |
| | POLYMETHYLSILSESQUIOXANE (7) | 2.4 | 2.4 |
| | NYLON-66 (8) | 8.0 | 8.0 |
| | TITANIUM DIOXIDE | 3.3 | 3.3 |
| | IRON OXIDES | 1.3 | 1.3 |
| | TALC | 13.9 | 18.9 |
| | DIMETHICONE/VINYL DIMETHICONE | 5.8 | 5.8 |
| | CROSSPOLYMER (9) | | |

(1) POLYETHYLENE AC 540 from HONEYWELL
(2) REGALITE R1100 from EASTMAN CHEMICAL
(3) KRATON G1657M from KRATON POLYMERS
(4) KSG-6 from SHIN-ETSU
(5) SB700 from MIYOSHI KASEI
(6) EXPANCEL 551 DE40 D42 from EXPANCEL
(7) TOSPEARL 145Afrom MOMENTIVE PERFORMANCE MATERIAL
(8) PULPE POLYAMIDE 12185 Taille 0.3 mm (POLYAMIDE PULP 12185 Size 0.3 mm) from UTEXBEL
(9) DOW CORNING 9506 POWDER from Dow Corning

The powder phase B is mixed using a Baker Perkins mixer.

Phase A is heated at 100° C. and stirred until a uniform mixture is obtained.

The two phases are introduced at the inlet of an extruder heated to 90° C. then cooled to 10° C. in the last barrel of the extruder.

The two compositions are applied easily to the face and make it possible to effectively mask the skin imperfections, in particular recessed areas of the skin, such as pores or acne scars, even deep ones. The composition according to the invention has a very good wear property over time on the skin, whereas the comparative composition readily detaches from the face or has highlight points after several hours.

### EXAMPLE 2

### Mascara

| | | Invention |
|---|---|---|
| Phase | INCI name | example |
| A | BEESWAX | 7.0 |
| | CARNAUBAWAX | 3.0 |
| | CERESIN WAX | 2.0 |
| | HYDROGENATED STYRENE/METHYL | 10.0 |
| | STYRENE/INDENE COPOLYMER (1) | |
| | HYDROGENATED STYRENE/BUTADIENE | 2.0 |
| | COPOLYMER (2) | |
| | ISOHEXADECANE | 11.0 |
| | ISODODECANE | 35.0 |
| | VINYL DIMETHICONE/LAURYLDIMETHICONE | 10.0 |
| | CROSSPOLYMER (3) | |
| B | SILICA (4) | 3.0 |
| | RAYON (5) | 1.0 |
| | BLACK IRON OXIDE | 5.0 |
| | TALC | 11.0 |

(1) REGALITE R1100 from EASTMAN CHEMICAL
(2) KRATON G1657M from KRATON POLYMERS
(3) KSG-42 from SHIN-ETSU
(4) SB700 from MIYOSHI KASEI
(5) FIBRE RCIBE N003 02M from CLAREMONT FLOCK

The mascara according to the invention is applied easily to the eyelashes with a suitable brush. It makes it possible to obtain a thick deposit which has good wear property on the eyelashes.

### EXAMPLE 3

### Lipstick

| | | Invention |
|---|---|---|
| Phase | INCI name | example |
| A | MICROCRYSTALLINE WAX | 4.7 |
| | ETHYLENE/ACRYLIC ACID COPOLYMER (1) | 2.3 |
| | HYDROGENATED STYRENE/METHYL | 8.0 |
| | STYRENE/INDENE COPOLYMER (2) | |
| | HYDROGENATED STYRENE/BUTADIENE | 1.0 |
| | COPOLYMER (3) | |
| | HYDROGENATED POLYISOBUTENE | 22.8 |
| | DIMETHICONE | 11.7 |
| | ISONONYL ISONONANOATE | 3.8 |
| | DIMETHICONE/VINYL DIMETHICONE | 9.7 |
| | CROSSPOLYMER (and) DIMETHICONE (4) | |
| B | MICA-IRON OXIDE NACRE (5) | 30.0 |
| | ACRYLATES COPOLYMER (6) | 0.2 |
| | DIMETHICONE/VINYL DIMETHICONE | 5.8 |
| | CROSSPOLYMER (7) | |

(1) POLYETHYLENE AC 540 from HONEYWELL
(2) REGALITE R1100 from EASTMAN CHEMICAL
(3) KRATON G1657M from KRATON POLYMERS
(4) KSG-6 from SHIN-ETSU
(5) COLORONA Sienna from MERCK
(6) EXPANCEL 551 DE40 D42 from EXPANCEL
(7) DOW CORNING 9506 POWDER from DOW CORNING

The powder phase B is mixed using a Baker Perkins mixer.

Phase A is heated at 100° C. and stirred until a uniform mixture is obtained.

The two phases are introduced at the inlet of an extruder heated to 90° C. then cooled to 10° C. in the last barrel of the extruder.

The composition is applied easily to the lips while colouring them, and effectively masks wrinkles.

### EXAMPLE 4

### Example of Transparent Smoothing Base

The following composition was prepared:

| | | Invention |
|---|---|---|
| Phase | INCI name | example |
| A | MICROCRYSTALLINE WAX | 8.00 |
| | ETHYLENE/ACRYLIC ACID COPOLYMER (1) | 4.00 |
| | HYDROGENATED STYRENE/METHYL | 8.50 |
| | STYRENE/INDENE COPOLYMER (2) | |
| | HYDROGENATED STYRENE/BUTADIENE | 0.50 |
| | COPOLYMER (3) | |
| | HYDROGENATED POLYISOBUTENE | 20.50 |
| | DIMETHICONE (4) | 12.30 |
| | ISONONYL ISONONANOATE | 4.00 |
| | DIMETHICONE/VINYL DIMETHICONE | 10.20 |
| | CROSSPOLYMER (and) DIMETHICONE (5) | |
| B | SILICA (6) | 8.00 |
| | ACRYLATES COPOLYMER (7) | 0.35 |
| | POLYMETHYLSILSESQUIOXANE (8) | 2.35 |
| | ALUMINUM STARCH OCTENYLSUCCINATE (9) | 8.00 |
| | TALC | 8.00 |
| | DIMETHICONE/VINYL DIMETHICONE | 5.30 |
| | CROSSPOLYMER (10) | |

(1) POLYETHYLENE AC 540 from HONEYWELL
(2) REGALITE R1100 from EASTMAN CHEMICAL
(3) KRATON G1657M from KRATON POLYMERS
(4) SILICONE FLUID 5CS from DOW CORNING
(5) KSG-6 from SHIN-ETSU
(6) SB700 from MIYOSHI KASEI
(7) EXPANCEL 551 DE40 D42 from EXPANCEL
(8) TOSPEARL 145A from MOMENTIVE PERFORMANCE MATERIAL
(9) DRY FLO PLUS from AKZO NOBEL
(10) DOW CORNING 9506 POWDER from DOW CORNING

The powder phase B is mixed using a Baker Perkins mixer.

Phase A is heated at 100° C. and stirred until a uniform mixture is obtained.

The two phases are introduced at the inlet of an extruder heated to 90° C. then cooled to 10° C. in the last barrel of the extruder.

The composition according to the invention contains 8.5% of adhesive resin and 32% of pulverulent phase.

Its elastic modulus is 70 000 Pa.

The composition was applied, using a spatula or with the fingers, to the whole of the face.

The composition adheres well to the skin and the skin imperfections, ie recessed areas of the skin (pores, wrinkles, scars) are transparently smoothed.

1. An anhydrous cosmetic composition, comprising, in a physiologically acceptable medium:
(i) a tackifying resin;
(ii) an organopolysiloxane elastomer; and
(iii) a liquid fatty phase.

2. The composition according to claim 1, wherein the tackifying resin (i) has a number-average molecular weight of less than or equal to 10 000 g/mol.

3. The composition according to claim 1, wherein the tackifying resin (i) is selected from the group consisting of rosin, a rosin derivative, a hydrocarbon-based resin, and mixtures thereof.

4. The composition according to claim 3, wherein the tackifying resin (i) is a hydrocarbon-based resin selected from the group consisting of an indene hydrocarbon-based resin, an aliphatic pentanediene resin, a mixed resins of pentanediene and of indene, a diene resin of cyclopentadiene dimers, a diene resin of isoprene dimers, and mixtures thereof.

5. The composition according to claim 1, wherein the tackifying resin (i) is an indene hydrocarbon-based resin.

6. The composition according to claim 1, wherein the organopolysiloxane elastomer (ii) is obtained by a crosslinking addition reaction (A) of a diorganopolysiloxane comprising at least two hydrogens each bonded to a silicon, and (B) of a diorganopolysiloxane comprising at least two ethylenically unsaturated groups bonded to silicon, optionally in the presence (C) of a platinum catalyst.

7. The composition according to claim 6, wherein the compound (A) is selected from the group consisting of a methylhydrogenpolysiloxane comprising trimethylsiloxy end groups, a dimethylsiloxane-methylhydrosiloxane copolymer comprising trimethylsiloxy end groups, and a dimethylsiloxane-methylhydrosiloxane cyclic copolymer.

8. The composition according to claim 6, wherein the compound (B) is a diorganopolysiloxane comprising at least two lower (for example C₂-C₄) alkenyl groups selected from the group consisting of a vinyl group, an allyl group and a propenyl group.

9. The Cosmetic according to claim 1, wherein the organopolysiloxane elastomer (ii) is free of polyoxyalkylene units and of polyglyceryl units.

10. The composition according to claim 1, wherein a weight ratio (by weight of active material) of the organopolysiloxane elastomer (ii) and the tackifying resin (i) ranges from 0.1 to 10.

11. The composition according to claim 1, wherein the liquid fatty phase (iii) comprises a hydrocarbon-based oil.

12. The composition according to claim 1, further comprising:
(iv) a pulverulent phase.

13. The composition according to claim 12, wherein a weight ratio of the liquid fatty phase (iii) and the pulverulent phase (iv) ranges from 0.1 to 10.

14. A method, comprising applying at least one layer of the composition according to claim 1 to a keratin material.

15. The composition according to claim 1, which is suitable for caring for, making up, or both, keratin materials.

16. The composition according to claim 5, wherein the tackifying resin (i) is a hydrogenated indene/methylstyrene/styrene copolymer.

17. The composition according to claim 12, wherein the pulverulent phase (iv) comprises a filler, a colorant, a fiber, or a mixture thereof.

18. The method according to claim 14, which is suitable for making up, non-therapeutically treating, or both, the keratin material.
